(19) United States
(12) Patent Application Publication
(10) Pub. No.: US 2026/0256375 A1
(43) Pub. Date: Sep. 3, 2026

(54) Endoscope Having Articulating Head With Flexural Platform for Measuring Impedance of Mucosa

(71) Applicant: Diversatek Healthcare, Inc., Milwaukee, WI (US)

(72) Inventors: Charles L. Lindsay, Monument, CO (US); Erik B. Holstad, Fredonia, WI (US); Matthew V. Pezzino, Denver, CO (US)

(21) Appl. No.: 18/846,838

(22) PCT Filed: Mar. 14, 2023
(86) PCT No.: PCT/US2023/015199

§ 371 Date: Sep. 13, 2024

Related U.S. Application Data
(60) Provisional application No. 63/319,901, filed on Mar. 15, 2022.

(51) Int. Cl. A61B 5/0538; A61B 1/00; A61B 5/00

(52) U.S. Cl. CPC A61B 5/0538; A61B 1/00097; A61B 1/0014; A61B 5/4211; A61B 5/4233; A61B 5/6847

(57) ABSTRACT

A mucosal impedance measuring device for measuring a pressure-controlled impedance of mucosa includes an endoscope having an elongated body extending to a scope end in which the scope end is articulable relative to the elongated body. The device further includes a platform flexibly supported at the scope end with a plurality of impedance measuring electrodes on the platform.

### CROSS-REFERENCE TO RELATED APPLICATIONS

This application claims the benefit of U.S. Provisional Patent Application No. 63/319,901 entitled “Endoscope Having Articulating Head with Flexural Platform for Measuring Impedance of Mucosa” filed Mar. 15, 2022, which is hereby incorporated by reference for all purposes as if set forth in its entirety herein.

### STATEMENT REGARDING FEDERALLY SPONSORED RESEARCH

Not applicable.

### BACKGROUND

Recently, links between the electrical properties of esophageal mucosal tissues and the health of these esophageal mucosal tissues have been identified and investigated. Specifically, it has been determined that, by the taking impedance measurements of the esophageal mucosa, the condition or health of those esophageal mucosa can be determined. For example, patients with gastroesophageal reflux disease (GERD) have damaged mucosa in regions of their esophagus that are evident when the impedance measurements of the damaged mucosa are compared to the measurements of healthy, undamaged mucosa. Importantly, such impedance measurements are taken with the impedance measuring electrodes being placed in controlled contact with the esophageal mucosa under regulated pressure. By ensuring good controlled pressure contact between the electrodes and the esophageal mucosa, meaningful impedance readings can be obtained which closely correspond to the health of the tissues based on empirical data.

Understanding this relationship between the impedance measurements of the esophageal mucosal and the health of the esophageal mucosa, unique devices have been designed to take such health-indicating impedance measurements. For example, U.S. Pat. No. 9,814,408 issued on Nov. 14, 2017 and U.S. Pat. No. 10,321,867 issued on Jun. 18, 2019, both of which are incorporated by reference for all purposes as if set forth in their entirety herein, disclose systems and methods involving the use of catheters and catheter systems that take pressure regulated impedance measurements of esophageal mucosa. Such devices are described as, for example, including catheters with associated balloons and impedance measuring electrodes which might be arranged in arrays or lines. During use of such a device, the patient is intubated with the catheter, the balloon is inflated to draw the electrodes into predicable and controlled contact with the esophageal mucosa, and impedance measurements are taken through the electrodes while the balloon remains inflated. The measured impedance can then be evaluated and compared to the known impedance values of heathy or unhealthy tissue. Furthermore, systems and methods have also been specifically disclosed in which an endoscope with an articulating scope end is used to press the electrodes against the mucosa by the action of articulation. See for example, WO Application Publication No. 2020/0176508 published on Sep. 3, 2020, which is incorporated by reference for all purposes as if set forth in its entirety herein. In such design, instead of a balloon or bladder generating the pressure, the end of the scope may be articulated such that the sides of the end carrying the electrodes are pressed into contact with the mucosa.

Such devices and impedance measurement methods of tissue are contemplated as permitting much faster identification of esophageal conditions as opposed to more traditional studies, which can last for multiple hours or even a few days.

### SUMMARY OF THE INVENTION

While catheterized systems of the type described above have been demonstrated to be able to collect useful impedance measurements of esophageal mucosa for time-efficient evaluation of esophageal mucosal health, some problems exist with such systems.

The current generation of mucosal integrity technology allows physicians to quickly evaluate the condition of the mucosa by measuring impedance over several centimeters of the esophagus to assist in the diagnosis of such conditions as gastroesophageal reflux disease (GERD), Non-Gerd or Normal, and eosinophilic esophagitis (EoE). However, these impedance measurements can be adversely affected by improper study site preparation, inability to remove excess fluid in the esophagus or cardia, or physical abnormalities in the esophagus, such as achalasia, which hamper proper sensor contact with the mucosal wall.

Still further, for endoscopes utilizing an articulating head to create the pressure rather than a balloon or bladder, the electrodes may not evenly contact the mucosa after the head articulates given the angular adjustment being made. For example, when the head is articulated relative to the rest of the elongated body, the electrodes or contact points closest to the axial end may come into contact with the mucosa at a greater pressure that those electrodes or contact points further from the axial end. For collecting pressure-regulated impedance measurements, this has the possibly to create some issues in the quality of the measurements taken, as the quality of the impedance measurement obtained is a function of the pressure between the electrodes and the mucosa.

Disclosed herein are improved solutions to address these issues. A new device structure is presented that is positioned on the tip of an endoscope and that includes a platform supporting electrodes that are flexibly supported relative to the end of the endoscope. This device can provide the physician with more control by allowing the physician the ability to visually place the sensors against the mucosal point of interest and quickly measure the mucosal integrity via impedance of the mucosa. Still further, the flexibly platform or rail with the electrodes or sensors provides various potential advantages. During placement of the electrodes or sensors against the mucosa, the platform carrying the sensors provides an initial asymmetrical pressure that forces unwanted liquid or gas to be removed from between the surface of the mucosa and the electrical sensors. This targeted and controlled pressure can substantially eliminate measurement error caused by liquid or gas entrapment. Then, during further use or application of pressure, the device can automatically adjust by virtue of the flexible platform carrying the impedance measuring electrodes, which bends to more evenly align with the surface of the mucosa to provide a controlled pressure over several centimeters of tissue. This flexure of the platform is apart from and independent of the articulation of the endoscopic tip and helps to position the platform and electrodes better and more evenly against the mucosa at the time of impedance measurement than might be the case with the articulation of the scope end alone with statically fixed electrodes mounted relative to that scope end.

Thus, the disclosed device and related method solves this problem by automatically balancing the contact pressure of all the mucosa impedance sensors contacting the mucosal wall. The system can electronically monitor and report the quality and stability of each impedance sensor, giving the physician real-time graphical feedback of the quality of their measurement.

According to one aspect, a mucosal impedance measuring device is provided for measuring a pressure-controlled impedance of mucosa. The device includes an endoscope having an elongated body extending to a scope end in which the scope end is articulable relative to the elongated body. The device further includes a platform flexibly supported at the scope end with a plurality of impedance measuring electrodes on the platform.

In some forms, the platform may be connected to a harness or sleeve that is secured to the endoscope. However, in other forms, the platform may be formed as part of the endoscope (e.g., formed integral therewith as a unitarily molded part thereof) and not an accessory secured or mounted thereto.

In some forms, upon articulation of the scope end during an endoscopy using the endoscope, the platform flexibly supported at the scope end and the impedance measuring electrodes thereon may be drawn into contact with the mucosa. Upon such contact, the platform may flex relative to the mucosa to equalize the pressure across the impedance measuring electrodes. That is to say, the pressure across the impedance measure electrodes against the mucosa may be more even given this ability for the platform to flex than if the platform was rigidly attached to the scope end of the endoscope and so the pressure between the electrodes and the mucosa is better regulated and controlled. This flexure may occur due to elastic deformation, for example, in the platform itself and/or a connection between the platform and a harness or a sleeve to which the platform is attached (or another support structure which may also be the endoscope itself). In some forms, this flexure may also result, at least in part, from some ability for the harness or sleeve to flex or shift relative to the endoscope to which the harness or sleeve is flexibly mounted. This flexing motion of the platform may create an application pressure that is initially asymmetrical and then subsequently balanced by the flexure, which may result in the working out of any fluid bubbles or pockets between the platform and the mucosa which, if present could impact the quality or validity of the impedance measurement taken.

In some forms, the platform may be flexibly supported by a “living hinge” or region of flexure that permits angular flexure of the platform and the impedance measuring electrodes received on that platform relative to the scope end of the endoscope. If the structure includes a harness or sleeve, then the living hinge may connect the platform to that harness or sleeve that is mounted to the endoscope. The living hinge may connect to a central portion of the platform. Such connection of the living hinge to the central portion of the platform may be at a position such that a smaller first length of the platform extends forward from the living hinge in the direction of a distal tip of the platform and a larger second length of the platform extends backward from the living hinge on the side of the living hinge away from the distal tip. In one form, the smaller first length may be approximately one-third of the overall length of the platform and the larger second length may be approximately two-thirds of the overall length of the platform. The larger second length of the platform that extends backward from the living hinge may connect to an elastic strip or tether that elongates upon angular flexure of the platform in which the second larger length of the platform is angled further upward. This strip or tether may help to “anchor” the rearmost end of the platform as well as inhibit or prevent the rearmost end of the platform from snagging on or pinching any tissue, especially upon withdrawal or extubation of the endoscope from a patient or on flexure of the platform. In some forms, the living hinge may include an opening therein through the living hinge and such opening may be suitable to receive wires or conductors extending from a sensor subassembly (i.e., from the electrodes) and to route them through the structure that flexibly supports the platform (e.g., the harness or sleeve). While a living hinge design is primarily contemplated, it is likewise considered that a two-material hinge (e.g., with pin and hinge halves, for example) could be employed instead of a living hinge.

In some forms, the impedance measuring electrodes may be part of a sensor subassembly. This sensor subassembly may be received in a recess of the platform.

In some forms, the plurality of impedance measuring electrodes may include four impedance measuring electrodes.

In some forms, the plurality of impedance measuring electrodes may be arranged along a line parallel to an axial direction of the platform. In such arrangement, the electrodes may be axially spaced from one another.

In some forms, the device may further include an impedance measuring system in electrical communication with the plurality of impedance measuring electrodes. The impedance measuring system may be configured to direct a current between the plurality of impedance measuring electrodes and through the mucosa and to measure the pressure-controlled impedance of the mucosa. Such pressure-controlled impedance can be used as a proxy or offer a metric or data for determining the health of the mucosa, especially when mapped over an area and used in conjunction with diagnosing a medical condition. To determine whether an impedance reading is accurate, the impedance measuring system may include software configured to determine whether the pressure-controlled impedance of the mucosa is a stable impedance measurement indicative of consistent pressure-regulated contact between the plurality of impedance measuring electrodes and the mucosa.

In some forms, the device may further include a plurality of conductors in which each of the conductors is in electrical communication with a corresponding one of the plurality of impedance measuring electrodes and in which the plurality of conductors extend from the impedance measuring electrodes for connection to, for example, the impedance measuring system.

According to another aspect, a method of measuring a pressure-controlled impedance of mucosa using the mucosal impedance measuring device described above and herein is provided. In the method, the scope end of the endoscope is articulated relative to the elongated body of the endoscope to draw the impedance measuring electrodes on the platform flexibly supported at the scope end of the endoscope into contact with the mucosa under an applied pressure from the articulating. During contacting the impedance measuring electrodes with the mucosa, the platform with the impedance measuring electrodes thereon is flexed to equalize the pressure across the impedance measuring electrodes. A current is conducted between the plurality of impedance measuring electrodes and through the mucosa and the pressure-controlled impedance of the mucosa is measured.

In some forms, the step of measuring the pressure-controlled impedance may involve using an impedance measuring system in electrical communication with the plurality of impedance measuring electrodes in which the impedance measuring system directs the current between the plurality of impedance measuring electrodes and through the mucosa and measures the pressure-controlled impedance of the mucosa.

In some forms, the method may further include the step of attaching a support or a harness proximate the scope end in which the support or the harness has the platform flexibly attached thereto. Such attachment may include the support or the harness being compressively connected to the scope end of the endoscope.

In some forms, the step of measuring the pressure-controlled impedance of the mucosa may include determining a quality of a reading of the pressure-controlled impedance of the mucosa. Determining the quality of the reading of the pressure-controlled impedance of the mucosa may, for example, involve assessing the stability of the reading over a time of measurement.

These and still other advantages of the invention will be apparent from the detailed description and drawings. What follows is merely a description of some preferred embodiments of the present invention. To assess the full scope of the invention, the claims should be looked to as these preferred embodiments are not intended to be the only embodiments within the scope of the claims.

### BRIEF DESCRIPTION OF THE FIGURES

FIG. 1 is a perspective view of a mucosal impedance measuring device showing an accessory having a harness or sleeve with a platform flexibly connected thereto that carries a plurality of impedance measuring electrodes and which is mounted on the scope end of an endoscope.

FIG. 2 is a cross-sectional side view of the mucosal impedance measuring device taken through line 2-2 of FIG. 1.

FIG. 3 is a side view of the mucosal impedance measuring device of FIG. 1 in an unstressed position and prior to forced contact with the mucosa.

FIG. 4 is a side view of the mucosal impedance measuring device of FIG. 3 after the head of the endoscope has been articulated to cause the platform carrying the electrodes into contact with the mucosal wall. This contact is illustrated to cause the platform carrying the electrodes to elastically flex relative to the tip of the endoscope as well as the sleeve or harness with which the platform is integrally formed to angle slightly with respect to the endoscope.

FIG. 5 is a side cross sectional view of the of the mucosal impedance measuring device of FIG. 1 after the head of the endoscope has been articulated to cause the platform carrying the electrodes into contact with the mucosal wall as in FIG. 4.

### DETAILED DESCRIPTION

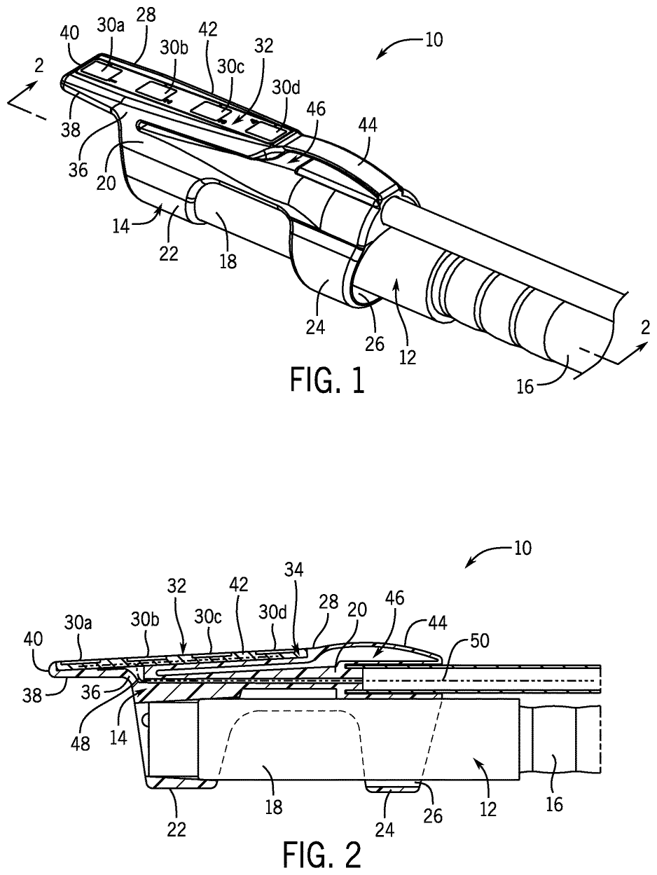

Referring to FIGS. 1 through 5, a mucosal impedance measuring device 10 and its constituent parts are illustrated. As illustrated, the mucosal impedance measuring device 10 includes an endoscope 12 and an accessory 14 attached to the endoscope 12 for electrical interrogation of mucosa as will be described in greater detail below. It is noted that, while the endoscope 12 and the accessory 14 are illustrated as separate components in the illustrated embodiment, it is contemplated that the structure of the accessory 14 might in other forms be integrated directly into the structure of an endoscope 12 and such combined structure is contemplated under the scope of this disclosure rather than the illustrated multi-component construction. Put differently, the so-named accessory could be made part of the endoscope itself or considered in some way to be part of the endoscope.

The endoscope 12 has an elongated body 16 extending to a scope end 18. The scope end 18 typically includes a camera and light on the axial end thereof for producing images of the inside of a patient or subject. The scope end 18 is articulable relative to the rest of the elongated body 16. This articulation both can be used to guide the endoscope 12 as it is inserted into the patient or subject and to direct the camera on the scope end 18 to capture video and/or images of interest within the body. The control of the articulation as well as the insertion/withdrawal of the endoscope 12 is typically manually performed by an operator such as a physician or technician to ensure that the scope end 18 is navigated appropriately.

In the form illustrated, the accessory 14 is supported by or mounted onto a forwardmost portion of the endoscope 12 at the scope end 18, which here is generally tubular in shape. The accessory 14 includes a body 20 which has on a lower side thereof a pair of axially-spaced loops including a forwardly-positioned loop 22 and a rearwardly-positioned loop 24 which serve as a harness or a sleeve to attach or secure the accessory 14 to the endoscope 12. The body 20 can be fabricated from a polymeric or elastomeric material in some forms such that one or more of the loops 22 and 24 are compressed around the endoscope 12 to secure the accessory 14 at the axial tip of the endoscope 12. To provide mechanical attachment between the two, the loop 22 can be sized such that a radially-inward facing surface of the loop 22 is approximately the same size or smaller than a radially-outward facing surface of the scope end 18 of the endoscope 12. In such case, with a small amount of temporary deformation of the body 20 and loop 22, the loop 22 can be compressively secured to the scope end 18 of the endoscope 12 by axial insertion thereon. Of course, this is but one way of attaching the accessory 14 to the scope end 18 of the endoscope 12 and, in constructions in which there is an accessory 14 with a body 20, other modes of attachment might also be used. For example, mechanically interlocking parts such as snaps or bayonet-type connections might be used to create mechanical engagement or fasteners of other types might form a connection between the two components. Still further, an adhesive, epoxy, or resin, whether permanent or temporary may be used to similar effect.

It is contemplated that one of the loops 22 and 24 might be slightly oversized to permit some off-axis deflection of the body 20 of the accessory 14 relative to the endoscope 12. See for, example, a gap 26 most apparent in FIGS. 1 and 2 is present between the endoscope 12 and the rearwardly-positioned loop 24 and the bottom end of the loop 24, which can provide some play or adjustability as depicted in FIG. 5 and as will be described in greater detail below.

Importantly and as best seen in FIG. 1, the mucosal impedance measuring device 10 includes a platform 28 flexibly supported at the scope end 18 with a plurality of impedance measuring electrodes 30a, 30b, 30c, and 30d on the platform 28. In the form illustrated, impedance measuring electrodes 30a, 30b, 30c, and 30d, are part of a sensor subassembly 32 which is received in a recess 34 of the platform 28. However, in other forms, the electrodes might be individually placed onto the platform 28 and not part of a subassembly. As best seen in the perspective view of FIG. 1, the impedance measuring electrodes 30a, 30b, 30c, and 30d are also axially spaced from one another and all situated along the axial length of the platform 28 and face radially outward relative to the endoscope 12.

In the form illustrated, the platform 28 is connected to the body 20 and more specifically, to the harness or sleeve, that is secured to the endoscope 12. Because the body 20 is composed of a flexible material, a relatively thin section connecting the platform 28 to the rest of the body 20 establishes a living hinge 36 or region of flexure and, thus, permits the platform 28 to pivot angularly relative to its orientation in FIG. 2, for example (note that FIG. 2 shows the platform 28 in an unstressed position; that is, at rest and without any applied forces). As used herein, the term “living hinge” is used to describe a region of elastically deformable material which permits the platform and the electrodes positioned thereon to be temporarily deflected under an applied load relative to the end of the endoscope, but to return to their original position after the applied load has been withdrawn.

As arranged in the illustrated embodiment, the living hinge 36 is positioned centrally over the length of the platform 28. As illustrated, the living hinge 36 is positioned approximately at a position to bifurcate the platform 28 into a smaller first length 38 of the platform 28 that extends forward from the living hinge 36 in the direction of a distal tip 40 of the platform 28 and a larger second length 42 of the platform 28 that extends backward from the living hinge 36 on the side of the living hinge away from the distal tip 40. The sensor subassembly 32 is roughly similarly bifurcated under this positioning of the living hinge 36 along the platform 28.

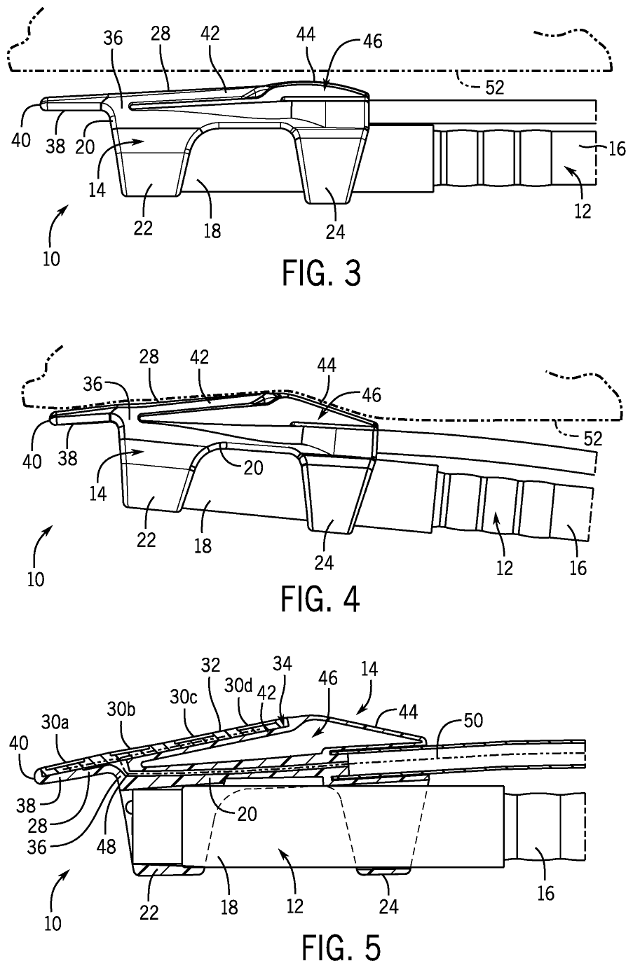

In addition to the living hinge 36 or region of flexure, there is an elastic strip or tether 44 that connects to the larger second length 42 of the platform 28 that extends backward from the living hinge 28. Comparing FIGS. 2 and 3 (unstressed) to FIGS. 4 and 5 (stressed), upon the flexure or movement of the platform 28 counterclockwise on the orientation depicted on the page, the tether 44 elongates. Both this tether 44 and the living hinge 28 will tend to draw the platform 28 back towards the unstressed position in FIGS. 2 and 3 from the deflected position, while still permitting temporary deformation under load to permit the angular deflection of the platform 28. Although angular rotation of the platform 28 in the counterclockwise direction is indicated above, it is contemplated that the platform 28 might also be made to rotate slightly in the clockwise direction in some circumstances; however, in such case the tether 44 would likely compress to some degree or bow to accommodate the deflection.

There is a space 46 between the platform 28 the body 20 and the tether 44 that is roughly triangular shaped that accommodates such flexure of the platform. The corners of that triangle are formed by the living hinge 36, the connection between the platform 28 and the tether 44, and the connection of the tether 44 to the body. Given this geometry the tether 44 can also serve as a wall that prevent the rearward end of the platform 28 from snagging on tissue during withdrawal of the device 10 from a patient, as lacking this tether 44, there may be the possibility of the rear end of the platform 28 catching on tissue in a fishhook-like fashion.

It can be seen in the cross sections of FIGS. 2 and 5, that there may be a small opening 48 extending through this living hinge 36 and then extending back through the body 20 of the accessory 14 in order to permit conductors (which denoted schematically by dashed lines 50) to be connected from the electrodes 30a, 30b, 30c, and 30d on the platform 28 to the elongated body 16 of the endoscope 12.

Turning now to the use case and method of operation of the device 10, the device 10 is designed to contact the platform 28 and electrodes 30a, 30b, 30c, and 30d disposed thereon with mucosa 52 and then take pressure regulated impedance measurements of the mucosa 52. In order to measure the impedance of mucosa 52 using an endoscope device such as that contemplated herein, the endoscope 12 is first inserted and navigated to the desired location within the patient. The camera of the endoscope 12 can then be used to determine that the endoscope 12 is generally placed within the patient as desired at the location where an impedance measurement of mucosa is desired. The device 10 prior to contact is schematically shown prior to contact with the mucosa 52 in FIG. 3. It should be appreciated that this image is schematic in nature and that in practice, the device 10 may be in some trivial contact with the mucosa 52 due to navigation through the body passage, although not in controlled contact/pressure circumstances. As is then depicted in FIG. 4, the scope end 18 of the endoscope 12 is articulated as denoted by the angular adjustment of the scope end 18 relative to the page. This articulation draws the leading tip 40 of the platform 28 and eventually the electrodes 30a, 30b, 30c, and 30d into contact with the mucosa 52.

With such articulation action, it is typically the case that the leading tip 40 of the platform 28 will engage the mucosa 52 under the greatest initial pressure and this will cause the flexure of the platform 28 relative to the scope end 18 (and the body 20 of the accessory 14 assuming the platform 28 is part of the accessory 14 and not the endoscope 12 directly). As depicted in FIG. 4, this pressure of the leading tip 40 on the mucosa 52 can cause the platform 28 to rotate at the living hinge 36 (in a counterclockwise direction based on the orientation depicted in FIG. 4) and/or for the rearwardly-position loop 24 to lift or move to some degree to adjust the accessory 14 relative to the scope end 18. In any event, the displacement from articulation results in contact that subsequently result in the platform 28 relatively pivoting relative to the scope end 18 in a manner that occurs in addition to any positional adjustment resulting from the articulation of the endoscope 12 alone. Under an applied load as in FIGS. 4 and 5, the elasticity in this region at the living hinge 36 permits angular flexure of the platform 28 and the impedance measuring electrodes 30a, 30b, 30c, and 30d received thereon relative to the scope end 18 to alter or adjust the angle of the platform 28 and the impedance measuring electrodes 30a, 30b, 30c, and 30d relative to the scope end 18 of the endoscope 12.

There are two things that can flow from this flexure of the platform 28. First, as the initial contact is at the distal tip 40 with the mucosa 52, the initial pressure over the length of the platform 28 is asymmetrical. So as the platform 28 tilts, any fluids or bubbles or other content between the platform 28 and the mucosa 52 may be worked out of the space between the two, thus providing a cleaner point of contact between the electrodes 30a, 30b, 30c, and 30d and the mucosa 52 which will, in turn, result in better electrical measurements. Second, the flexure of the platform 28 also helps to even out, equalize or balance the pressure over the axial length of the platform 28. Put differently, whereas the pressure between the electrodes at the axial end of the platform and the mucosa would be comparably higher near the distal tip than away from the tip in a non-flexing platform, a flexing platform permits the pressure over the length of the platform to level out.

After the platform 28 has flexed and the electrodes 30a, 30b, 30c, and 30d are in pressure-regulated contact with the mucosa 52, then an impedance measuring system in electrical communication therewith can be used to control the flow of a current through the conductors 50 into the electrodes 30a, 30b, 30c, and 30d in order to measure the impedance of the mucosa 52 between sets of the electrodes. The impedance measuring system can include a processor, controller, a current source, and circuity for measuring the impedance between the impedance measuring electrodes 30a, 30b, 30c, and 30d. For example, it can include a current production source which can be used to run current from one electrode to another (and through mucosa, as will be described below) and circuity for measuring the impedance between the electrodes (and, again, of the mucosa therebetween).

It is contemplated that the impedance measuring system can not only include electronics for obtaining impedance measurements, but also software and/or hardware for determining whether the measured impedance measurements are valid. Since, as explained above and from the patent and application incorporated by reference, the impedance measurements of mucosa are only valid if taken under controlled pressure and are sufficiently stable (meaning that good consistent contact is made between the electrodes and the mucosa), it is contemplated that the impedance measuring system can include testing logic to evaluate and confirm with the end user whether an obtained impedance measurement of mucosa is a good and valid measurement or includes stability issues or absolute impedance values that are indicative of an improper reading due to bad or inconsistent contact between the electrodes and the mucosa. For example, it is contemplated that impedance might be measured over a predetermined window of time (perhaps, a few hundred milliseconds or various seconds) and the signal of the impedance measured over time analyzed to determine whether the impedance is stable and within expected ranges for either healthy or unhealthy mucosa.

Still further, it is contemplated that there could be pressure-sensing elements attached to electrodes or between the electrodes that independently and electronically confirm stable pressure contact exists between the electrodes and mucosa when the scope end is articulated and the platform is deflected. Such pressure-sensing elements and information therefrom might be used separately from or in combination with the software/hardware analysis of the impedance signal to assess the validity of an impedance measurement.

Such methods may also be employed in pull-out studies in which a reading is taken and then the device is pulled out some distance before taking another reading. Such pull-out studies permit a length of tissue to be mapped over distance in excess of the overall length of the electrodes on the device by taking and combining various measurements at known positions iteratively.

Still further, it is contemplated that the mucosal impedance measuring device 10 may be implemented in such a way that it can be used to map the passageway it is inserted into, specifically in the case of the gastrointestinal tract or colon, and display visual information relating to the gastrointestinal tract or colon dimensionally, to the location of the endoscope (particularly the scope end) within the gastrointestinal tract or colon, and/or to impedance measurements that have been taken relating to tissue health. Such visual depiction or display can occur on a monitor or other viewing device attached to the mucosal impedance measuring device and/or the endoscope. For example, during an insertion of the endoscope into and through the gastrointestinal tract, one or more spatial locations of an endoscope may be recorded. This recording of this positional data may be done manually for example, by the operator of the endoscope inputting information about the position of the scope end of the endoscope (i.e., providing the software information about when initial insertion is occurring, when the scope is at a bend between one region and another or at some other predefined location, and so forth). Such recording may be automated in part or in whole for example by software that prompts the operator for input of certain information or that monitors and analyzes the manner in which the endoscope is inserted and the manner in which is navigated through the passageways of the patient to detect these conditions in a “smart” manner. In some instances, it is contemplated the position determinations may be made in whole or in part using imaging from the camera or other modes of interrogation to determine the position and path of the scope end of the endoscope as it is inserted. With this positional information available, a location of the endoscope may be visually depicted within a generated image of the gastrointestinal tract produced by the method. For example, the operator or physician performing the endoscopy may map the interior of a colon during an initial insertion process by entering information about certain data points (e.g., when a curve from one region to another region of the colon is being made) which may be depicted on a computer monitor or display, for example. With the colon physically mapped and depicted, the operator will then be able to visually see the location of the scope end of the endoscope during further examination as the software accounts for the length of insertion or withdrawal of the endoscope once the mapping has occurred and presents this position on the mapped image.

It is contemplated that such visual depiction might be two-dimensional or even three-dimensional. Three-dimensional depiction may require some additional input stream, such as potentially a video stream that calculates diameter of the patient's passageway locally or may involve some other reading collected from the endoscope indicative of diameter of the colon in the localized region.

Still further, all of the mucosal impedance collecting steps described above with respect to the general operation of the mucosal impedance measuring device 10 may be performed contemporaneously with mapping or after mapping. Such mucosal impedance collection may be used to map a single point or multiple points (perhaps involving a length) of the colon with respect to health of the tissue. Indeed, when electrodes are accessible on different sides of the endoscopic tip, it may even be possible to take multiple peripheral measurements at a particular insertion depth of the endoscope for three-dimensional inspection of the tissue. While the general understanding in the state of the art is that tissue at a particular insertion depth should be equally healthy on all sides, multiple readings may be able to be made to collect various data points which can then be averaged and/or used to determine whether there is a difference in tissue over the periphery that may be of interest. While a single flexible platform is illustrated in the depicted embodiment, it is contemplated that a plurality of flexible platforms similar in structure to the platforms described above could be arranged at various angular positions about the axis of the scope end (i.e., at different angular positions about the periphery). It is well contemplated that two, three or four (or even more) flexible platforms similar to the platform above could be employed on a single endoscope as part of the accessory or the endoscope itself, with the limiting factor for the number of platforms being size constraints as well as decreasing utility once a sufficient degree of angular coverage is provided. With such a multi-platform construction, regardless of the direction of actuation of the tip of the endoscope relative to the body and passage, at least one of the platforms could be drawn into contact with the mucosa for a reading. Similarly, the scope might be articulated in a number of directions at a particular insertion depth to collect impedance measurements of the tissue on the various sides without having to twist the endoscope.

Accordingly, such visualization of the gastrointestinal tract may include not just information about the physical dimensions of the gastrointestinal tract, but also provide indications of tissue health from the mucosal impedance measurements. After one or more collected mucosal impedance measurements are taken, they then may then be visually mapped on corresponding region(s) of the map of the colon, for example. By visually plotting the mucosal impedance measurement(s) on a visual depiction of the colon, it may be easier for the operator or patient to visualize comprehend where the readings are being taken and, in the case of multiple readings, visualize more holistically the health of the tissue over a length of interest of the colon and understand the nature of any irregularity by providing better physical context, depicted visually.

When graphically depicting the mucosal impedance on the visual depiction of the gastrointestinal tract, it is considered that the impedance readings may be color-coded to improve understanding by the viewer. For example, measurements that are taken as being indicative of healthy tissue may be depicted as green, while unhealthy tissue depicted as red. Still further, color gradients could be used to depict either the magnitude of the reading (e.g., light green for marginally healthy tissue and dark green for strongly healthy tissue.

Still further, it is contemplated that apart from merely mapping tissue health using color and/or number, it is possible that quality of the reading (e.g., the stability of the reading) might also be mapped physically on the visualized depiction. In this way, the operator may also be able to better assess whether certain regions were more difficult than others to collect measurements from and, as a result of this difficultly in collection, if the measurements from that area may need to be recollected or be examined more closely or skeptically. Still further, it may be possible to provide other layers not just for health of tissue base on impedance or quality of reading, but for other conditions of interest, such as for example the average diameter of the localized region of the colon if such information has been collected.

Returning now to the structure of the endoscope and accessory for the endoscope, it is contemplated that in addition to the structures and methods described above, various other modifications and additions might be feasible to facilitate authentication of the device and/or ensure proper use. As one example, when the device is provided as an accessory to an endoscope, the accessory structure might house a small chip (for example, in the structure of the support) which could provide authentication information to the impedance measuring system to confirm that the accessory device is genuine and/or provide calibration information relating to the specific accessory so that, when that calibration information is accounted for by the impedance measuring system, the impedance measurements taken with the accessory are accurate. Each device may be factory calibrated to ensure each device has a consistent impedance point of reference that is consistent to the balloon device. Still further, in such case that the impedance measuring electrodes are tightly integrated into the endoscope, such authentication information and/or calibration data might similarly be housed in the structure of the endoscope. It is also contemplated that such a chip or memory could store information about the number of times that the accessory (or endoscope, if integrated) has been used to ensure both that the accessory (or endoscope) is being properly used and has not been fouled in some way since its calibration. For example, the device may be engineered for one-time use or N-time uses and the chip may hold information about whether the use or N-time uses has occurred or not; it is contemplated that, if the use or uses have occurred, then the impedance measuring system may provide the user with an indication that the device cannot be used without first replacing the accessory (or endoscope, if integrated).

The present disclosure has described one or more preferred embodiments, and it should be appreciated that many equivalents, alternatives, variations, and modifications, aside from those expressly stated, are possible and within the scope of the invention.

What is claimed is:

1. A mucosal impedance measuring device for measuring a pressure-controlled impedance of mucosa, the device comprising:
an endoscope having an elongated body extending to a scope end in which the scope end is articulable relative to the elongated body; and
a platform flexibly supported at the scope end with a plurality of impedance measuring electrodes on the platform.

2. The mucosal impedance measuring device of claim 1, wherein, upon articulation of the scope end during an endoscopy using the endoscope, the platform flexibly supported at the scope end and the impedance measuring electrodes thereon are drawn into contact with the mucosa and the platform is able to flex to relative to the mucosa to equalize the pressure across the impedance measuring electrodes.

3. The mucosal impedance measuring device of claim 1, wherein the platform is connected to a harness or sleeve that is secured to the endoscope.

4. The mucosal impedance measuring device of claim 1, wherein the platform is flexibly supported by a living hinge that permits angular flexure of the platform and the impedance measuring electrodes received thereon relative to the scope end.

5. The mucosal impedance measuring device of claim 4, wherein the living hinge connects the platform to a harness or sleeve that is secured to the endoscope.

6. The mucosal impedance measuring device of claim 4, wherein the living hinge connects to a central portion of the platform.

7. The mucosal impedance measuring device of claim 6, wherein the living hinge connects to the central portion of the platform at a position such that a smaller first length of the platform extends forward from the living hinge in the direction of a distal tip of the platform and a larger second length of the platform extends backward from the living hinge on the side of the living hinge away from the distal tip.

8. The mucosal impedance measuring device of claim 7, wherein the larger second length of the platform that extends backward from the living hinge connects to an elastic strip or tether that elongates upon angular flexure of the platform in which the second larger length of the platform is angled further upward.

9. The mucosal impedance measuring device of claim 8, wherein the elastic strip or tether avoids a possibility of an otherwise exposed terminal rear end of the platform fish-hooking or snagging on mucosa during an extubation of the endoscope.

10. The mucosal impedance measuring device of claim 6, wherein the living hinge includes an opening therein through.

11. The mucosal impedance measuring device of claim 1, wherein the impedance measuring electrodes are part of a sensor subassembly.

12. The mucosal impedance measuring device of claim 11, wherein the sensor subassembly is received in a recess of the platform.

13. The mucosal impedance measuring device of claim 1, wherein the plurality of impedance measuring electrodes include four impedance measuring electrodes.

14. The mucosal impedance measuring device of claim 1, wherein the plurality of impedance measuring electrodes are arranged along a line parallel to an axial direction of the platform.

15. The mucosal impedance measuring device of claim 1, further comprising an impedance measuring system in electrical communication with the plurality of impedance measuring electrodes in which the impedance measuring system is configured to direct a current between the plurality of impedance measuring electrodes and through the mucosa and to measure the pressure-controlled impedance of the mucosa.

16. The mucosal impedance measuring device of claim 15, wherein the impedance measuring system includes software configured to determine whether the pressure-controlled impedance of the mucosa is a stable impedance measurement indicative of consistent pressure-regulated contact between the plurality of impedance measuring electrodes and the mucosa.

17. The mucosal impedance measuring device of claim 1, further comprising a plurality of conductors in which each of the conductors is in electrical communication with a corresponding one of the plurality of impedance measuring electrodes and in which the plurality of conductors extend from the impedance measuring electrodes.

18. A method of measuring a pressure-controlled impedance of mucosa using the mucosal impedance measuring device of claim 1, the method comprising:
articulating the scope end of the endoscope relative to the elongated body of the endoscope to draw the impedance measuring electrodes on the platform flexibly supported at the scope end of the endoscope into contact with the mucosa under an applied pressure from the articulating;
during contacting the impedance measuring electrodes with the mucosa, flexing the platform with the impedance measuring electrodes thereon to equalize the pressure across the impedance measuring electrodes; and
conducting a current between the plurality of impedance measuring electrodes and through the mucosa and measuring the pressure-controlled impedance of the mucosa.

19. The method of claim 18, wherein the step of measuring the pressure-controlled impedance involves using an impedance measuring system in electrical communication with the plurality of impedance measuring electrodes in which the impedance measuring system directs the current between the plurality of impedance measuring electrodes and through the mucosa and measures the pressure-controlled impedance of the mucosa.

20. The method of claim 18, further comprising the step of attaching a support or a harness proximate the scope end in which the support or the harness has the platform flexibly attached thereto.

21. The method of claim 20, wherein the support or the harness is compressively connected to the scope end of the endoscope.

22. The method of claim 18, wherein the step of measuring the pressure-controlled impedance of the mucosa includes determining a quality of a reading of the pressure-controlled impedance of the mucosa.

23. The method of claim 22, wherein determining the quality of the reading of the pressure-controlled impedance of the mucosa involves assessing the stability of the reading over a time of measurement.
